(19) United States
(12) Patent Application Publication
(10) Pub. No.: US 2008/0213351 A1
(43) Pub. Date: Sep. 4, 2008

(54) Compositions and methods for dosing liposomes of certain sizes to treat or prevent disease

(75) Inventors: Wendi V. Rodrigueza, Ann Arbor, MI (US); Charles L. Bisgaier, Ann Arbor, MI (US)

Correspondence Address: JONES DAY, 222 EAST 41ST ST, NEW YORK, NY, 10017, US

(73) Assignee: Esperion LUV Development, Inc.

(21) Appl. No.: 12/070,949

(22) Filed: Feb. 21, 2008

Related U.S. Application Data
(63) Continuation of application No. 10/403,402, filed on Mar. 31, 2003, now abandoned.
(60) Provisional application No. 60/370,409, filed on Apr. 5, 2002.

(51) Int. Cl. A61K 9/127; A61P 9/10; A61P 3/10

(52) U.S. Cl. 424/450

(57) ABSTRACT

The present invention relates to pharmaceutical compositions comprising liposomes and methods of using such liposomes to prevent, treat, or manage a variety of diseases and/or bodily conditions. The liposomes may comprise large unilamellar vesicles (LUVs) alone or in combination with multilamellar vesicles (MLVs), small unilamellar vesicles (SUVs), or other therapeutics. The invention relates to liposomes having certain diameters administered to patients using specific doses and/or dosing regimens.

This application is a continuation of U.S. patent application Ser. No. 10,403,402, filed Mar. 31, 2003, which claims the benefit of U.S. Provisional Application No. 60/370,409, filed Apr. 5, 2002, incorporated by reference herein in its entirety.

### FIELD OF THE INVENTION

The present invention relates to pharmaceutical compositions comprising liposomes and methods of using such liposomes to prevent, treat, or manage a variety of diseases and/or bodily conditions. The liposomes may comprise large unilamellar vesicles (LUVs) alone or in combination with multilamellar vesicles (MLVs), small unilamellar vesicles (SUVs), or other therapeutics. The invention relates to liposomes having certain diameters administered to patients using specific doses and/or dosing regimens.

### BACKGROUND OF THE INVENTION

Atherosclerosis is the leading cause of death in the United States. Atherosclerosis is the formation of plaques in arterial walls that can occlude the vessel lumen and obstruct blood flow through the vessel. The plaques can also rupture and lead to thrombus formation even in a blood vessel without a critical stenosis and can lead to occlusion of blood vessels and the obstruction of blood flow. Morbidity and mortality generally occur through end organ damage and organ dysfunction resulting from ischemia. The most common forms of ischemic end organ damage are myocardial infarction and cerebrovascular accidents. Disability or death often result from these vascular events. Even atherosclerosis-related ischemia that does not permanently injure myocardium or leads to minor myocardial damage is responsible for significant morbidity in the form of angina pectoris and congestive heart failure. Other organs, such as the kidneys, the intestines, and the spinal cord, may also be injured by atherosclerotic occlusions. Further, in diseases such as aortic aneurysms, atherosclerotic arteries may cause clinical symptoms independent of end organ dysfunction. Arteriosclerotic lesions are plaques that form by accumulation of cholesterol, cholesterol esters, and phospholipids, proliferation of smooth muscle cells, the diapedesis of monocytes including their transformation into macrophages and foam cells in the intima of major arteries. Lipid contributes a major portion of the plaque volume (generally 30-65% dry weight). Small, ARTERIOSCLEROSIS, 8: 103-129 (1988). In fact, the risk of developing arteriosclerosis is directly related to the concentration of certain forms of plasma cholesterol.

Other diseases and conditions associated with abnormally high lipid levels include acute coronary syndromes, stable angina, unstable angina, inflammation, vascular inflammation, dermal inflammation, coronary heart disease (CHD), ventricular arrythmias, peripheral vascular disease, peripheral occlusive disease, intermittent claudication, transient ischemic attacks, restenosis, decreased need for revascularization, coagulation disorders, ischemia, cardiovascular ischemia, ischemia unrelated to cardiovascular disease such as ischemia-reperfusion injury, thrombocytopenia, pancreatitis, non-alcoholic steatohepatitis (NASH), diabetic neuropathy, retinopathy, painful diabetic neuropathy, claudication, psoriasis, critical limb ischemia, impotence, hyperlipidemia, hyperlipoproteinemia, hypoalphalipoproteinemia, hypertriglyceridemia, any stenotic condition leading to ischemic pathology, diabetes, ichtyosis, stroke, vulnerable plaques, lower-limb ulceration, severe coronary ischemia, lymphomas, cataracts, endothelial dysfunction, xanthomas, vascular disease, vascular disease that results from smoking and diabetes, carotid and coronary artery disease, congestive heart failure, regress and shrink established plaques, Alzheimer's Disease, combinations of surgical procedures that result in endothelial injury, endothelial damage as a result of surgical procedures, morbidity associated with vascular disease, ulcerations in the arterial lumen, restenosis as a result of balloon angioplasty, and subindications of the foregoing.

Lipids, including cholesterol, are generally insoluble in aqueous plasma. Plasma lipids are carried by soluble lipoprotein complexes. These lipoprotein complexes consist of an inner core of non-polar lipids (cholesteryl esters and triglycerides) and a surface layer of amphipathic proteins and polar lipids (phospholipids and non-esterified cholesterol). Different proteins (apolipoproteins) are present in the surface coat of different lipoprotein complexes (lipoproteins). The different lipoproteins perform different functions in lipid metabolism.

Five classes of lipoproteins are known. Some lipoproteins carry triglycerides and cholesterol from the liver to peripheral tissues while others transport lipids to the liver. Cholesterol may be metabolized in the liver to bile salts that are excreted, thus lowering total body cholesterol. Two lipoproteins, low density lipoproteins (LDL) and high density lipoproteins (HDL), have a high degree of association with the development of atherosclerosis. LDL has a high cholesterol concentration, delivers lipids to cells of peripheral tissues, and is associated with a high risk of atherosclerosis. HDL also has a relatively high cholesterol concentration, but carries lipids to the liver for metabolism into bile salts and is associated with decreasing the risk of developing atherosclerosis.

Cholesterol metabolism and homeostasis is the result of a complex equilibrium between free sterol in the cell and in plasma. Phillips et al., BIOCHIM. BIOPHYS. ACTA, 906: 223-276 (1987). Delivery of cholesterol to cells occurs via the receptor-mediated LDL pathway, selective uptake through scavenger receptor B-I, and by passive exchange of sterol between plasma membranes and lipoproteins. Only tissues that produce steroid hormones and bile acids can metabolize cholesterol. In order to prevent accumulation of excess free sterol in remaining peripheral tissues there is a reverse transport of cholesterol from plasma membranes into HDL and lipoprotein-like particles. HDL transports excess cholesterol to the liver where it can either be exported in the bile as unesterified cholesterol, processed into bile salts for excretion, esterified and stored in the liver, or incorporated into very low density lipoproteins (VLDL) to re-enter the lipoprotein pool.

The passive exchange of cholesterol between cells and lipoproteins occurs via the diffusion of sterol molecules across the aqueous space. Phillips et al., supra, and Schroeder et al., EXP. BIOL. MED., 196: 235-252 (1991). Net cellular efflux occurs if the chemical potential of free cholesterol is lower in the plasma than in the cells so that sterol leaves the membrane following its activity gradient. Under these conditions, it has been shown that cholesterol-ester-loaded cells, which are morphologically characteristic of early atherosclerotic lesions, not only lose cholesterol, but promote ester hydrolysis, resulting in the reduction of intracellular deposits of this lipid. Small, ARTERIOSCLEROSIS, 8: 103-129 (1988). Moreover as mentioned above, there is epidemiological evidence that conditions which might be expected to enhance reverse cholesterol transport (low plasma cholesterol concentrations, or increased HDL concentrations) are correlated with reduced risk of premature atherosclerosis and may give rise to plaque regression.

Characteristically, plaques are associated with ulceration of the vessel intima. The lipid-containing plaques grow in the ulcerations projecting friable masses into the arterial lumen. The plaques may also injure and weaken the smooth muscle media of the vessel. As plaque formation progresses, more central regions of the plaques are shielded from the circulation. Extensive plaque formation also causes concentric or eccentric constriction of the vessel at the plaque site.

Presently, the most effective treatment of atherosclerosis is prevention. There is evidence that the progression and accumulation of lipids in lesions can be halted when plasma LDL concentrations are kept to near normal levels. Reynolds, Circulation, 79: 1146-1148 (1989). Current preventive management of atherosclerotic disease has focused on the use of drugs in conjunction with dietary restrictions to regulate plasma cholesterol levels. Moreover, antioxidant therapies which suppress the formation and uptake of modified LDL particles by the cells of the arterial wall are also proving beneficial. Chisolm, CLIN. CARDIOL., 14: 25-30 (1991). However, while hypocholesterolemic drugs induce favorable plasma cholesterol changes which appear to slow the progression of atherosclerosis, they do not generally induce conditions that promote the efflux and removal of atheroma cholesterol. Clearly, in order to achieve significant regression of atheroma and lessen lumen obstruction, these space occupying lipids must be mobilized. Present evidence suggests that processes which stimulate the efflux of extra hepatic cell cholesterol and transport it to the liver for excretion, reverse cholesterol transport (RCT), are important events in the prevention of atherosclerosis. Gwynne, Clin. CARDIOL., 14: 17-24 (1991).

Current therapeutic modalities of arteriosclerosis are generally divided into surgical and medical management. Surgical therapy may entail vascular graft procedures to bypass regions of occlusion (e.g., coronary artery bypass grafting), removal of occluding plaques from the arterial wall (e.g., carotid endarterectomy), or percutaneously cracking the plaques (e.g., balloon angioplasty). Surgical therapies carry significant risk and only treat isolated lesions. Atherosclerotic plaques downstream from the treated lesion may continue to obstruct blood flow. Surgical therapies also do not limit the progression of atherosclerosis and are associated with the late complication of restenosis.

Medical therapy is directed to reducing other risk factors related to vascular disease (e.g., smoking, diabetes, and hypertension) and lowering forms of serum cholesterol that are associated with the development of atherosclerosis as described above. While medical therapies may slow the progression of plaque formation, plaque regression is relatively rare. Therefore, symptomatic atherosclerosis often requires both surgical and medical treatment.

Paradoxically, intravenous infusion of phospholipids and liposomes has been shown to produce regression of atherosclerotic plaques although serum lipid levels are transiently elevated. Williams et al., PERSPECT. BIOL. MED., 27: 417-431 (1984). In some instances, however, cholesterol associated with development and progression of atherosclerosis may increase following liposome administration.

Previous studies investigating phospholipid-induced mobilization of cholesterol in vivo have employed multilamellar or sonicated liposome vesicles. Liposome size is a key characteristic in clearance kinetics and is one of several reasons why sonicated vesicles have been expected to represent the bilayer structure best suited to enhance reverse cholesterol transport. Sonication reduces multilamellar vesicles (MLV) to ‘limit size’ vesicles. These systems exhibit the minimum radius of curvature that can be adopted by the bilayer configuration without disruption. For example, the minimum size egg phosphatidylcholine liposome that can be generated is typically about 30-nm diameter, often classified as a small unilamellar vesicle (SUV). For a given liposome composition, it is generally assumed that the smaller the particle diameter the greater the circulation half-life (Gregoriadis and Senior, LIFE SCI., 113: 183-192 (1986)). Consequently, it was expected that SUV composed of phosphatidylcholine would circulate longer than larger liposomes, and therefore mobilize more cholesterol. Furthermore, packing constraints experienced by phospholipids in SUV, (due to the acute radius of curvature) gives rise to an instability that can result in fusion, Hope et al., CHEM. PHYS. LIPIDS, 40: 89-107 (1986), as well as an increased tendency to assimilate with lipoproteins. See, e.g., Scherphofet al., BIOCHIM. BIOPHYS. ACTA, 542: 296-307 (1978) and Krupp et al., BIOCHIM. BIOPHYS. ACTA, 72: 1251-1258 (1976). Therefore, it was expected that SUV would produce a greater number of HDL-like particles, thus promoting efflux of sterol from peripheral tissues. Supporting this expectation, liposomes having diameters of 50-80 nm have been reported to optimize sterol mobilization and plaque regression. European Patent Publication No. 0461559 A2.

U.S. Pat. No. 5,746,223 (the '223 patent), entitled “Method of Forcing The Reverse Transport of Cholesterol From A Body Part While Avoiding Harmful Disruptions Of Hepatic Cholesterol Homeostasis,” U.S. Pat. No. 6,080,422 (the '422 patent), entitled “Methods of Angioplasty and Cardiac Catheterization,” and U.S. Pat. No. 6,139,871 (the '871 patent), entitled Liposome Compositions And Methods For The Treatment of Artherosclerosis, each of which is incorporated herein by reference in their entirety, disclose (inter alia) the use of LUVs to induce the reverse transport of cholesterol from peripheral tissues, to treat atherosclerosis, and for use in angioplasty procedures.

More specifically, the '223 and '422 patents disclose the use of LUVs with a diameter up to 1000 nm (larger liposomes may be “less efficient”), with claims reciting larger than about 50, 80, or 100 nm diameters and the specification disclosing a preferable diameter of 120 nm in one embodiment. The doses administered in the '223 and '422 patents range from 10-1600 mg/kg per dose. In more specific embodiments the dose is less than 600 mg/kg per day and in other particular embodiments the dose is a bolus of 300 mg/kg administered to rabbits at one, three, and five days.

The '871 patent discloses the use of LUVs having a diameter ranging from 100-150 nm with a preferable diameter of 125 nm. The '871 patent also discloses administering LUVs in a range of about 100-150 mg/kg, “usually” about 200 to 750 mg/kg, and “most usually” about 280 to 420 mg/kg in multiple treatments, “generally weekly” for about 4-16 weeks, “usually” about 10 weeks. The '871 patent states that the concentration of the LUVs in the carrier may vary, but generally the concentration will be about 20-200 mg/ml, usually about 50-150 mg/ml, and most usually about 100 mg/ml.

Recently, two studies were conducted using ETC-588 (liposomes made from 1-palmitoyl, 2-oleoyl phosphatidylcholine, a proprietary product of Esperion Therapeutics, Inc.). ETC-588 sequesters cholesterol and other exchangeable lipids from vascular and peripheral tissue (a process known as mobilization) resulting in transient increases in serum cholesterol that return to predose levels after ETC-588 delivers its cholesterol load to the liver for processing or excretion. Mobilization and regression of experimentally induced atherosclerosis has been shown in pre-clinical models using a liposome size and composition very similar to ETC-588. ETC-588 acutely increases cholesterol flux within the body and enhances reverse lipid transport pathways.

The first study assessed the safety and tolerability of ETC-588 in healthy volunteers with single doses of 20, 60, 150, 300, and 600 mg/kg of ETC-588 liposomes. ETC-588 liposomes (200 mg/ml) were infused intravenously at 10 ml per minute using an infusion pump. ETC-588 in plasma was assayed as phospholipid (PL). Total and unesterified cholesterol (UC) and PL were assayed by standard, automated methods. Subjects were allocated to treatment groups according to a randomization schedule. Safety and tolerability, laboratory data, vital signs and adverse events were summarized at each time point and for pre-dose to pose-dose change using descriptive statistics. Pharmacokinetic and pharmacodynamic measures were summarized using descriptive statistics. The results indicated that although cholesterol mobilization occurred across a wide range of doses the efficiency varied. In addition, three out of three subjects had elevated liver function tests at 600 mg/kg and one individual had a serious adverse event indicated by increased liver function enzymes. Other adverse events included headache, dizziness, nausea, and fatigue.

The second study assessed the safety and tolerability of ETC-588 in healthy volunteers who received a total of 4 doses given at 3 day intervals. Doses administered were 100, 150, 200, 250, and 300 mg/kg. ETC-588 liposomes (200 mg/ml) were infused intravenously at 10 ml per minute using an infusion pump. ETC-588 in plasma was assayed as phospholipid (PL). Total and unesterified cholesterol (UC) and PL were assayed by standard, automated methods. Subjects were allocated to treatment groups according to a randomization schedule. Safety and tolerability, laboratory data, vital signs and adverse events were summarized at each time point and for pre-dose to post-dose change using descriptive statistics. Pharmacokinetic and pharmacodynamic measures were summarized using descriptive statistics. The results indicated that cholesterol mobilization increased in a dose-dependent fashion, although efficiency varied. In addition, dosing at 3 day intervals for a total of 4 doses appeared to cause an undesirable accumulation of ETC-588. As in the first study, other adverse events included headache, dizziness, nausea, and fatigue. Furthermore, these dosing studies, were conducted solely on healthy patients and not on patients with disease or other undesirable bodily conditions.

Therefore, a need exists to determine safe, effective, and non-toxic doses and dosing regimens in diseased patients that prevents or reduces any unwanted side effects. In addition, there is a need to determine more optimal dosing regimens using optimally sized liposomes to treat patients suffering from the diseases and/or bodily conditions disclosed herein or other diseases or conditions.

### SUMMARY OF THE INVENTION

The present invention relates to pharmaceutical compositions and methods of administering liposomes which may comprise LUVs alone or in combination with MLVs, SUVs and/or other therapeutics used to prevent, treat or manage a variety of diseases and bodily conditions including, but not limited to: arteriosclerosis including atherosclerosis, phlebosclerosis or any venous condition in which deposits of plaques containing cholesterol or other material are formed within the intima or inner media of veins, acute coronary syndromes, angina including stable angina and unstable angina, inflammation or inflammatory diseases including but not limited to vascular inflammation and dermal inflammation, congestive heart failure, coronary heart disease (CHD), hypertension, coronary ventricular arrythmias, surraventricular arrythmias, peripheral vascular disease, fatal myocardial infarction, non-fatal myocardial infarction, ischemia including cardiovascular ischemia, myocardium hybernation, transient ischemic attacks, ischemia unrelated to cardiovascular disease including ischemia-reperfusion injury such as injury due to hip surgery, knee surgery, organ transplant or PTCA, coronary reperfusion, restenosis, peri-operative (PCI) ischemic events, decreased need for revascularization, reduced infarct area, coagulation disorders, thrombocytopenia, deep vein thrombosis, pancreatitis, non-alcoholic steatohepatitis (NASH), diabetic neuropathy, retinopathy, diabetic neuropathy, psoriasis, critical limb ischemia, claudication, impotence, prostate cancer, hyperlipidemia, hyperlipoproteinemia, hypoalphalipoproteinemia, hypertriglyceridemia, any stenotic condition leading to ischemic pathology, diabetes including both Type I and Type II, ichtyosis, stroke, vulnerable plaques, vulnerable plaque rupture, Alzheimer's disease, lower-limb ulceration, severe coronary ischemia, lymphomas, cataracts, endothelial dysfunction, xanthomas, end organ dysfunction, vascular disease, vascular disease that results from smoking and diabetes, carotid and coronary artery disease, regress and shrink established plaques, combinations of surgical procedures that result in endothelial injury, endothelial damage as a result of surgical procedures, morbidity associated with vascular disease, ulcerations in the arterial lumen, restenosis as a result of balloon angioplasty, and subindications of the foregoing.

The compositions and methods of the present invention may be used to increase HDL levels, increase low HDL levels, decrease LDL levels, decrease high LDL levels, temporarily increase LDL levels, decrease triglycerides levels, increase or decrease the level of other lipids, increase plaque stability or decrease the probability of plaque rupture, increase or decrease vasodilation, treat or prevent inflammation, treat or prevent inflammatory diseases or an inflammatory response, strengthen or stabilize smooth muscle and vessel intima, stimulate efflux of extracellular cholesterol for transport to the liver, modulate immune responses, mobilize cholesterol from atherosclerotic plaques, and modify any membrane, cell, tissue, organ, and extracellular region and/or structure in which compositional and/or functional modifications would be advantageous. The compositions and methods of the present invention also encompass topical applications and wound healing.

The invention encompasses dosing regimens wherein specific doses of liposomes, especially liposomes within a particular size range, are administered at specific time intervals and specific doses to achieve reduction of cholesterol and/or treatment of disease while reducing or avoiding adverse effects or unwanted effects. Thus, methods of administering liposomes, methods of reducing total and LDL cholesterol by the administration of liposomes, methods of raising the level or increasing the efficacy of HDL cholesterol by the administration of liposomes, and methods of dosing liposomes in patients in need thereof are described herein in detail.

The vesicle particles (or liposomes) optimize cholesterol efflux from atherosclerotic plaques. The vesicle particles may be bound to an apolipoprotein, typically apolipoprotein AI or AII and often contain at least one phospholipid, such as phosphatidylcholine or phosphatidylglycerol. The compositions generally comprise liposomes and a pharmaceutically acceptable carrier. In a preferred embodiment the liposomes are cholesterol-free prior to administration. The liposomes used within the novel dosing regimens may have a diameter of any size. In addition, the liposomes may have an average diameter of any size with any standard deviation or size distribution.

In a separate and preferred embodiments the liposomes have an average diameter between 50 to 250 nanometers (nm) with any standard deviation or size distribution. In separate preferred embodiments the liposomes have an average diameter between 50 to 250 nm with a distribution of ±50%, preferably ±40%. In separate preferred embodiments, the liposomes have an average diameter between 100 to 140 nm with any standard deviation or size distribution. In separate preferred embodiments, the liposomes have an average diameter between 100 to 140 nm with a distribution of ±50%, preferably ±40%. In separate preferred embodiments, the liposomes have an average diameter between 110 to 120 nm with any standard deviation or size distribution. In separate preferred embodiments, the liposomes have a diameter between 110 to 120 nm with a distribution of ±50%, preferably ±40%. In separate preferable embodiments, the liposomes have an average diameter between 100 and 200 nm with any standard deviation or size distribution. In particular embodiments, the liposomes have an average diameter size between: 100 and 110 nm, 110 and 120 nm, 120 and 130 nm, 130 and 140 nm, 140 and 150 nm, 150 and 160 nm, 160 and 170 nm, 170 and 180 nm, 180 and 190 nm, or between 190 and 200 nm with any standard deviation or size distribution, preferably the size distribution is between ±40% and ±50%. In particular embodiments the liposomes utilized are ETC-588 (a proprietary product of Esperion Therapeutics, Inc.).

Methods for treating the above-identified diseases and bodily conditions are also provided which may be employed therapeutically or prophylactically. The methods generally comprise administering the compositions of the present invention to mammals, preferably humans, having any of the above-mentioned diseases or bodily conditions. Often, the compositions will be serially administered over a period of time. The compositions may be administered orally or parenterally. Generally, the compositions will be administered parenterally, preferably intravenously or the administration may be intramuscular, subcutaneous, intraperitoneal, intrathecal, intra-arterial, via lymphatics, via infusion, intravascular, and administration via a chronically indwelling catheter or via an acutely placed catheter via syringe or push administration. In other embodiments, the administration may be sublingual, buccal, mucosal, topical, rectal, vaginal, intra-arterial, transdermal, via infusion, via syringe, or via push administration. In a particular embodiment, the compositions of the present invention are administered topically to prevent or treat inflammation or to aid in the healing of wounds.

The methods of the present invention encompass the administration of liposomes in single or divided doses between 20 mg/kg and 600 mg/kg, preferably between 50 mg/kg and 600 mg/kg, and more preferably between 50 mg/kg and 300 mg/kg to a patent in need thereof. In a separate preferred embodiment liposomes are administered in single or divided doses between 100 mg/kg and 200 mg/kg, preferably between 150 mg/kg and 200 mg/kg to a patient in need thereof. In particular embodiments, the vesicle particles are administered in a single or divided doses between 110 mg/kg and 120 mg/kg, 120 mg/kg and 130 mg/kg, 130 mg/kg and 140 mg/kg, 140 mg/kg and 150 mg/kg, 150 mg/kg and 160 mg/kg, 160 mg/kg and 170 mg/kg, 170 mg/kg and 180 mg/kg, 180 mg/kg and 190 mg/kg, or between 190 mg/kg and 200 mg/kg to a patient in need thereof.

In specific embodiments, the vesicle particles are administered in a single or divided dose in one or more intervals ranging from once a day, once every 2 days, once every 3 days, once every 4 days, once every 5 days, once every six days, once a week, once every two weeks, once every three weeks, once a month, once every two months, once every three months, once every four months, once every five months, and once every 6 months, once every 7 months, once every 8 months, once every nine months, once every ten months, once every eleven months, and once a year, or otherwise administered at predetermined time intervals for a predetermined treatment period. In a preferred embodiment the time interval between doses during a course of therapy is once a week. Preferred treatment periods for a course of therapy may span 1 day, 2 days, 3 days, 4 days, 5 days, 6 days, one week, two weeks, three weeks, four weeks, five weeks, six weeks, seven weeks, eight weeks, nine weeks, ten weeks, eleven weeks, twelve weeks, thirteen weeks, fourteen weeks, four months, five months, six months, seven months, eight months, nine months, ten months, eleven months, one year, two years, three years, four years, or five years. In a preferred embodiment, the treatment period for a course of therapy is no longer than 14 weeks. In other preferred embodiments, liposomes are administered at 4-7 day intervals with either 1-4, 1-8, or 1-14 doses given with each course of therapy. Dose regimes also include continuous infusion treatment that may include the use of a primer dose followed by a maintenance dose. Patients treated with such compositions and according to such methods may be of any age and may be afflicted with one or more of the diseases or bodily conditions enumerated above and/or other diseases and conditions.

### BRIEF DESCRIPTION OF THE FIGURES

FIG. 1A shows the size distribution in a batch of large unilamellar liposomes produced by extrusion.

FIG. 1B shows the size distribution in a batch of large unilamellar liposomes of approximately 200 nm produced by homogenization.

FIG. 1C shows the size distribution in a batch of ETC-588 liposomes produced by extrusion.

FIG. 2 illustrates the levels of liposome and unesterified cholesterol after doses of liposome were administered every 4 days.

FIG. 3 illustrates the levels of liposome and unesterified cholesterol after doses of liposome were administered every 7 days.

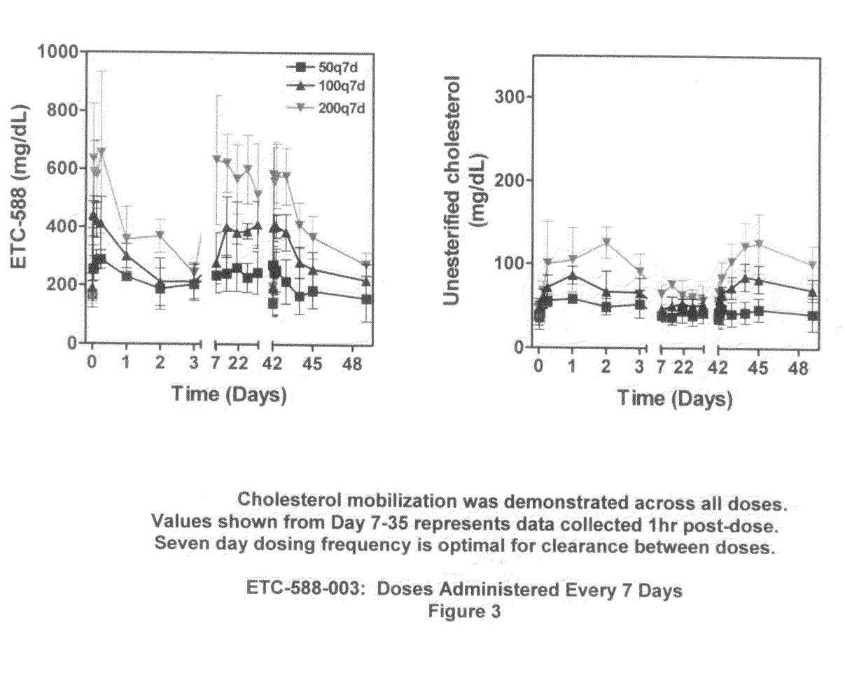

### USEFUL DEFINITIONS

As used herein, “drug” is meant to indicate a synthetic compound suitable for therapeutic use without associated bound carriers, adjuvants, activators, or co-factors. “Drug” does not include natural or endogenous apolipoproteins, lecithin-cholesterol acyltransferase, or albumin. “Liposome”, “vesicle” and “liposome vesicle” will be understood to indicate structures having lipid-containing membranes enclosing an aqueous interior. The structures may have or one more lipid membranes unless otherwise indicated, although generally the liposomes will have only one membrane. Such single layered liposomes are referred to herein as “unilamellar”. The term “LUVs” refers to large unilamellar vesicles, the term “SUVs” refers to small unilamellar vesicles, and the term “MLVs” refers to multilamellar vesicles. As used herein, the term “treating atherosclerosis” encompasses performing a therapeutic intervention that results in reducing the cholesterol content of at least one atherosclerotic plaque or prophylactically inhibiting or preventing the formation or expansion of an atherosclerotic plaque.

### DETAILED DESCRIPTION OF THE INVENTION

The present invention is based, in part, on the discovery that specific doses and dosing regimens used to deliver liposomes, particularly liposomes of specific size play critical roles in optimizing the removal or cholesterol from peripheral tissues and/or the metabolism of cholesterol removed from atherosclerotic plaques by such liposomes. The novel methods of the invention encompass the treatment or prevention of disease or the symptoms thereof while reducing or avoiding adverse effects, e.g., toxic side effects or unwanted effects. In addition, contrary to previous descriptions of liposome therapy, the inventors of the present invention have discovered that liposomes may be used to prevent, treat or manage a variety of diseases and bodily conditions besides atherosclerosis and other indications previously described for liposome therapy.

In one embodiment, the invention encompasses a method of dosing liposomes to a patient in need thereof, which method comprises administering liposomes with an average diameter of 100 nm and preferably less than 250 nm, to a patient having a disease treatable by cholesterol reduction in a single or divided dose administered every 4 to 7 days of from about 100 mg/kg to about 250 mg/kg, preferably from about 100 mg/kg to about 200 mg/kg. The patients to be treated, the liposomes to be used in the present invention and the specific doses and dosing regimens are discussed in detail below.

1. Liposomes

The inventors of the present invention have discovered that liposomes with an average diameter greater than 100 nm and preferably ranging from 100 nm to 140 nm, more preferably from 110 nm to 120 nm are optimal for cholesterol removal from the system. In general, the superior action of liposomes greater than 100 nanometers in diameter may be explained by the micro-anatomy of the liver. When circulating in the liver, large liposomes (as used herein, liposomes greater than 100 nm in diameter) may be cleared predominantly by the Kupffer cells that line the sinusoidal openings. The Kupffer cells transfer cholesterol to hepatocytes for excretion in the bile or for re-utilization. Small liposomes (as used herein, liposomes smaller than 100 nm) may directly access hepatocytes without (or with limited) prior processing by the Kupffer cells. Because for a fixed dose there may be more small liposomes infused than larger sized particles, hepatocytes may be acutely exposed to a relatively high concentration of small liposomes and their accumulated cholesterol. The inventors of the present invention have discovered that liposomes with a diameter of between 100 nm and 140 nm, preferably 110 nm and 120 nm are optimal because they are both cleared predominantly by the Kupffer cells and are more effective in mobilizing cholesterol than either smaller or larger sized liposomes.

In addition to and separate from the liposome size distribution, the inventors have discovered that in diseased patients certain doses and dose regimens are both safer and more effective in treating diseases. In such methods and the compositions used within the methods, one may use liposomes of any size or liposomes having any average diameter with any standard deviation or size distribution. Indeed, it is preferred that liposomes of the optimal size be used within the novel dosing regimens or within the preferred doses.

In specific embodiments, the liposomes have an average diameter greater than 100 nm regardless of the standard deviation or size distribution. In separate preferred embodiments the liposomes have an average diameter between 50-250 nanometers (nm) with any standard deviation or size distribution. In separate preferred embodiments the liposomes have an average diameter between 50-250 nm±40-50%. In separate preferred embodiments, the liposomes have an average diameter between 100-140 run with any standard deviation or size distribution. In separate preferred embodiments, the liposomes have an average diameter between 100-140 nm±40-50%. In separate preferred embodiments, the liposomes have an average diameter between 110-120 run with any standard deviation or size distribution. In separate preferred embodiments, the liposomes have an average diameter between 110-120 nm±40-50%. In separate preferable embodiments, the liposomes have an average diameter between 100 and 200 nm with any standard deviation or size distribution. In particular embodiments, the liposomes have an average diameter size between: 100 and 110 nm, 110 and 120 nm, 120 and 130 nm, 130 and 140 nm, 140 and 150 nm, 150 and 160 nm, 160 and 170 nm, 170 and 180 nm, 180 and 190 nm, or between 190 and 200 nm with any standard deviation or size distribution, preferably the size distribution is between ±40% and ±50%. In a preferred separate embodiment, the liposomes utilized are ETC-588 (a proprietary product of Esperion Therapeutics, Inc.) having an average diameter between 100 nm and 140 nm±40-50% after manufacture.

The invention also contemplates the use of liposomes with an average diameter greater than 50 nm and preferably greater than 100 nm and less than 250 nm which when administered, does or does not raise LDL serum concentrations. The invention also contemplates the use of liposomes with an average diameter greater than 50 nm and preferably greater than 100 nm and less than 250 nm which when administered does or does not lower LDL serum concentrations. In some instances the liposomes will not be bound to another molecule such as a drug or protein. In other instances, the liposomes of the present invention will be bound, combined and/or administered in combination with: (1) proteins and peptides bound to liposomes such as lipid binding proteins including peptides, paraoxonase, lipoprotein lipase, lecithin cholesterol acyl transferase, phospholipid transfer protein, Apo A-I and mimetics or variants of ApoA-I; (2) small acceptors including HDL, synthetic and/or recombinant HDL particles, synthetic and/or recombinant HDL particles made with apolipoproteins ApoA-I or ApoA-I mimetics; (3) cardiovascular agents including small molecules, statins, aspirin, clopidogrel, beta-blockers, glycemic control and/or anti-diabetic agents, antihypertensive agents, heparin, nitrates, IIb/IIIa inhibitors, ACE inhibitors, beta-blockers, fibrates, calcium channel blockers, and/or bile acid sequestrants; (4) and antidiabetic (and/or glycemic control) pharmacotherapy including but not limited to insulin and oral agents. In addition, large liposomes may be administered alone or in combination with multilamellar vesicles and/or small unilamellar vesicles. Examples of proteins and peptides (such as lipid binding proteins including peptides, Apo A-I and mimetics or variants of A-I ) are described in U.S. Pat. Nos. 6,004,925, 6,046,166, 6,037,323, 6,287,590, 6,329,341, and 6,265,377 all of which are incorporated by reference herein in their entirety. Examples of small molecules that moderate HDL, LDL or cholesterol levels are described in U.S. patent application Ser. Nos. 09/540,740, 09/540,739, and 09/540,738 all of which are incorporated by reference herein in their entirety.

Persons of skill will appreciate that the liposomes in the compositions of the present invention may be synthesized by a variety of methods, such as described in, e.g., U.S. Pat. Nos. 4,186,183; 4,217,344; 4,261,975; 4,485,054; 4,774,085; 4,946,787; 5,726,157; 5,746,223; 5,843,474; 5,448,435; 5,853,402; 6,080,422, 6,312,719; 6,139,871, PCT Publication No. WO 91/17424, Deamer and Bangham, BIOCHIM. BIOPHYS. ACTA, 443: 629-634 (1976); Fraley et al., PROC. NATL. ACAD. SCI. USA, 76: 3348-3352 (1979); Hope et al., BIOCHIM. BIOPHYS. ACTA, 812: 55-65 (1985); Mayer et al., BIOCHIM. BIOPHYS. ACTA, 858: 161-168 (1986); and Williams et al., PROC. NATL. ACAD. SCI., 85: 242-246 (1988), each of which is incorporated herein by reference in their entirety. Suitable methods include, e.g., sonication, extrusion, high pressure/homogenization, microfluidization, detergent dialysis, calcium-induced fusion of small liposome vesicles, and ether-infusion methods, all well known in the art.

Generally, the liposomes are most conveniently generated by sonication and extrusion procedures. Briefly, lipid is mixed with physiological saline and buffer. In one embodiment, a chloroform solution of lipid is vortexed and the solvent removed under a steady stream of N₂. The sample is dried under a high vacuum. The resulting dry lipid film is rehydrated in 150 mM NaCl and 20 mM [4-(2-hydroxyethyl)]-piperazine-ethanesulfonic acid (Hepes, pH 7.4). This generally produces multilamellar liposomal vesicles. Unilamellar vesicles are prepared by sonication or extrusion.

Sonication is generally performed with a tip sonifier, such as a Branson tip sonifier, in an ice bath. Typically, the suspension is subjected to several sonication cycles. Extrusion may be carried out by membrane extruders, such as the Lipex Biomembrane Extruder. Defined pore size in the extrusion filters may generate unilamellar liposomal vesicles of specific sizes. The liposomes may also be formed by extrusion through an asymmetric ceramic filter, such as a Ceraflow Microfilter, commercially available from the Norton Company, Worcester Mass. ETC-588 liposomes (a proprietary product from Esperion Therapeutics, currently being evaluated in clinical trials) are produced by extrusion. The current method involves hydrating 1-palmitoyl-2-oleoyl phosphatidylcholine (POPC) with phosphate-buffered saline and pushing these through membrane filters until an average diameter of about 100 to 140 nm is achieved, and more preferably until an average diameter of about 110 to 120 nm is achieved. Single or multiple passes may be made through the membrane filters. In one specific embodiment, 2-10 passes are made, preferably 2-5 passes are made. In addition, the filter membranes may be the same or different sizes (i.e., there may be a gradient of membranes). The resulting product is called ETC-588. Examples of preparing liposomes of specific sizes by extrusion are described in U.S. Provisional Patent Application No. 60/326,032, filed on Sep. 28, 2001, which is incorporated herein by reference in its entirety.

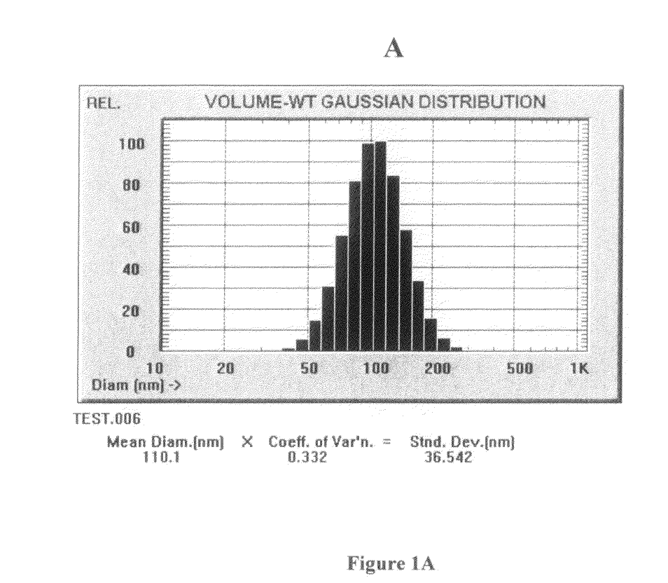

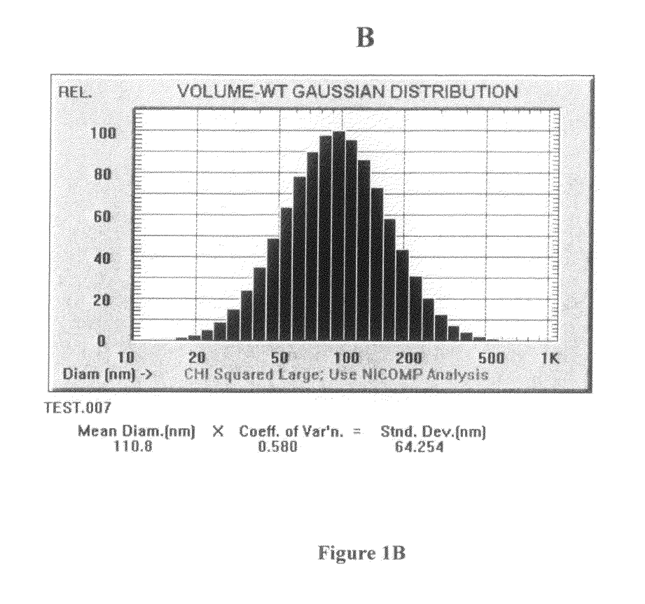

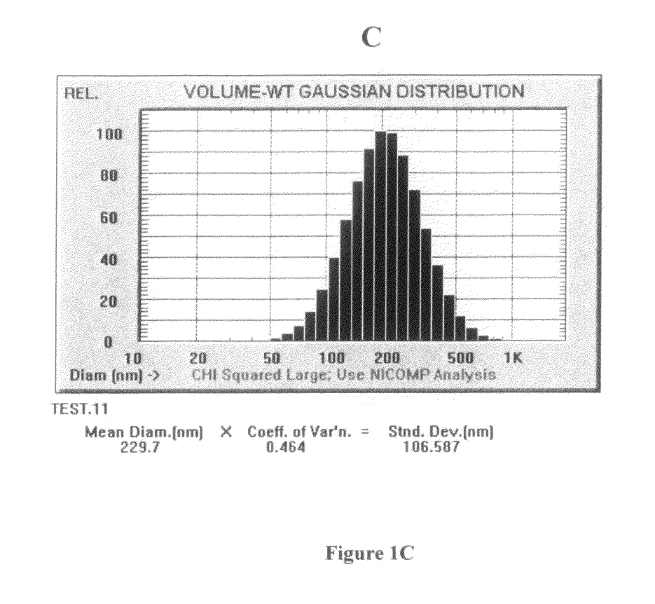

Other ways of producing liposomes with the same average diameter include homogenization or microfluidization, however the distribution of particles is larger. See FIG. 1A versus FIG. 1B. Using current technology, the biological effect particles produced by homogenization or microfluidization are different from those produced by extrusion. The clearance kinetics is slightly faster and area under the curve measurements for cholesterol mobilization are slightly less indicating that these particles are less efficacious than extruded liposomes even when animals are administered the equivalent dose on a mg phospholipid/kg basis.

The size of the liposomal vesicles may be determined by quasi-electric light scattering (QELS) as described in Bloomfield, ANN. REV. BIOPHYS. BIOENG., 10: 421-450 (1981), incorporated herein by reference in its entirety. Average liposome diameter may be reduced by sonication of formed liposomes. Intermittent sonication cycles may be alternated with QELS assessment to guide efficient liposome synthesis. The liposomes may be composed of a variety of lipids. Generally, the liposomes will be composed of at least one phospholipid, typically egg phosphatidylcholine, egg phosphatidylglycerol, distearoylphosphatidylcholine, or distearoylphosphatidylglycerol. Many embodiments of the present invention will include more than one phospholipid.

Other phospholipids suitable for formation of liposomes that can be used within the compositions or methods described herein include, but are not limited to, phosphatidylcholine, phosphatidylglycerol, lecithin, beta, gamma-dipalmitoyl-alpha-lecithin, sphingomyelin, phosphatidylserine, phosphatidic acid, N-(2,3-di(9-(Z)-octadecenyloxy))-prop- 1-yl-N,N,N-trimethylammonium chloride, phosphatidylethanolamine, lysolecithin, lysophosphatidylethanolamine, phosphatidylinositol, cephalin, cardiolipin, cerebrosides, dicetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleoyl-phosphatidylcholine, oleoyl-palmitoyl phosphatidylcholine, palmitoyloleoyl phosphatidylcholine, lipid which is in a liquid-crystalline phase at 37° C., mixtures thereof, and the like. In a preferred embodiment, the liposomes are composed of palmitoyloleoyl phosphatidylcholine. Non-phosphorus containing lipids may also be used in the liposomes of the compositions of the present invention. These include, e.g., stearylamine, docecylamine, acetyl palmitate, fatty acid amides, and the like. Additional lipids suitable for use in the liposomes of the present invention are well known to persons of skill in the art and are cited in a variety of well known sources, e.g., MCCUTCHEON'S DETERGENTS AND EMULSIFIERS and MCCUTCHEON's FUNCTIONAL MATERIALS, Allured Publishing Co., Ridgewood, N.J., both of which are incorporated herein by reference in their entirety.

Generally, it is desirable that the liposomes be composed of lipids that are liquid-crystalline at 37° C., often at 35° C., and even 32° C. Liposomes in the liquid-crystalline state typically accept cholesterol more efficiently than liposomes in the gel state. As patients typically have a core temperature of about 37° C., liposomes composed of lipids that are liquid-crystalline at 37° C. are generally in a liquid-crystalline state during treatment and, therefore, optimize removal of cholesterol from plaques.

Preferably the liposomes used within the methods and compositions are cholesterol-free.

2. Pharmaceutical Compositions

The pharmaceutical compositions of the present invention may comprise a pharmaceutically acceptable carrier or diluent. Many pharmaceutically acceptable carriers may be employed in the compositions of the present invention. Generally, normal saline will be employed as the pharmaceutically acceptable carrier. Other suitable carriers include, e.g., water, buffered water, 0.4% saline, 0.3% glycine, and the like, including glycoproteins for enhanced stability, such as albumin, lipoprotein, globulin, etc. These compositions may be sterilized by conventional, well known sterilization techniques. The resulting aqueous solutions maybe packaged for use or filtered under aseptic conditions and lyophilized, the lyophilized preparation being combined with a sterile aqueous solution prior to administration. The compositions may contain pharmaceutically acceptable auxiliary substances required to approximate physiological conditions, such as pH adjusting and buffering agents, tonicity adjusting agents and the like, for example, sodium acetate, sodium lactate, sodium chloride, sodium phosphate, potassium chloride, calcium chloride, etc.

The concentration of liposomes may vary. In particular embodiments the concentration of liposomes may range from about 20 mg/ml to about 1000 mg/ml, preferably between 50-300 mg/ml, more preferably between 50-200 mg/ml, and more preferably between 100-200 mg/ml. In separate preferable embodiments the concentration of liposomes may range from about 1-20 mg/ml, 20-30 mg/ml, 30-40 mg/ml, 40-50 mg/ml, 50-60 mg/ml, 60-70 mg/ml, 70-80 mg/ml, 80-90 mg/ml, 90-100 mg/ml, 100-110 mg/ml, 110-120 mg/ml, 120-130 mg/ml, 130-140 mg/ml, 140-150 mg/ml, 150-160 mg/ml, 160-170 mg/ml, 170-180 mg/ml, 180-190 mg/ml, or 190-200 mg/ml. In certain preferable embodiments, the concentration will be about 50 mg/ml, 100 mg/ml, 150 mg/ml, 200 mg/ml, 250 mg/ml, or 300 mg/ml. Persons of skill may vary these concentrations to optimize treatment with different liposomal components or of particular patients. For example, the concentration may be increased to lower the fluid load associated with treatment. This may be particularly desirable in patients having atherosclerosis-associated congestive heart failure or severe hypertension. Alternatively, liposomes composed of irritating lipids may be diluted to low concentrations to lessen inflammation at the site of administration.

The liposomes may optionally be bound to a variety of proteins and polypeptides to increase the rate of cholesterol transfer or the cholesterol-carrying capacity of the liposomes. Binding of apolipoproteins to the liposomes is particularly useful. As used herein, “bound to liposomes” or “binding to liposomes” indicates that the subject compound is covalently or non-covalently bound to the surface of the liposome or contained, wholly or partially, in the interior of the liposome. Apolipoprotein A_{I}, apolipoprotein A_{II}, and apolipoprotein E will generally be the most useful apolipoproteins to bind to the liposomes. These apolipoproteins promote transfer of cholesterol and cholesteryl esters to the liver for metabolism. Lecithin-cholesterol acyltransferase is also useful for metabolizing free cholesterol to cholesteryl esters. Liposomes in the pharmaceutical compositions of the present invention maybe bound to molecules of apolipoprotein A_{I}, apolipoprotein A_{II}, apolipoprotein E, and lecithin-cholesterol acyltransferase, singly or in any combination and molar ratio. Additional proteins or other non-protein molecules may also be useful to bind to the liposomes to enhance liposome stability or half-life and the like. These include, e.g., cholesterol, polyethylene glycol-linked phospholipid and gangliosides, sterols, alkylsulfates, ammonium bromide, albumin, and the like.

In specific embodiments, pharmaceutical compositions for use in accordance with the present invention may be formulated in a conventional manner using one or more physiologically acceptable carriers comprising excipients and auxiliaries which facilitate processing of the active compounds into preparations which can be used pharmaceutically. In specific embodiments, such formulations include stabilizers and/or antioxidants. Proper formulation is dependent upon the route of administration chosen. The liposomes of the present invention may be in lyophilized forms, liquid forms, frozen forms, or powder forms. In particular embodiments the liposomes of the present invention are in powder form, preferably lyophilized form, more preferably frozen form, and most preferably liquid form.

For particular embodiments, the liposomes of the invention may be formulated in aqueous solutions for injection, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art. In specific embodiments, topical or transdermal formulations include stabilizers and/or antioxidants.

In other embodiments, the compounds can be formulated readily for oral administration by combining the liposomes with pharmaceutically acceptable carriers well known in the art. Such carriers enable the compounds of the invention to be formulated as tablets, pills, dragees, capsules, push-fit capsules made of gelatin, soft or sealed capsules made of gelatin and plasticizer, liquids, gels, syrups, slurries, suspensions and the like, for oral ingestion by a patient to be treated. In addition, stabilizers may be added. All formulations for oral administration should be in dosages suitable for such administration.

In particular embodiments relating to buccal administration, the compositions may take the form of drops, tablets or lozenges formulated in conventional manner. The liposomes may be formulated for parenteral administration by injection, e.g., by bolus injection or continuous infusion as described above. In several embodiments the compounds may be administered by continuous infusion intravenously, intramuscularly or subcutaneously. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Additionally, suspensions of liposomes may be prepared as appropriate oily injection suspensions. Aqueous injection suspensions may contain substances which increase the viscosity of the suspension, such as sodium carboxymethyl cellulose, glycerol, sorbitol, or dextran. Optionally, the suspension may also contain suitable stabilizers or agents which increase the solubility of the liposomes to allow for the preparation of highly concentrated solutions.

Alternatively, the active ingredient may be in powder or lyophilized form for constitution or reconstitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

In addition to the formulations described previously, the liposomes may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials or ion exchange resins, or as sparingly soluble derivatives.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

3. Methods of Preventing, Treating or Managing Certain Diseases and Other Bodily Conditions with Liposomes

Also provided are methods for preventing, treating or managing a variety of diseases and bodily conditions including, but not limited to: arteriosclerosis including atherosclerosis, phlebosclerosis or any venous condition in which deposits of plaques containing cholesterol or other material are formed within the intima or inner media of veins, acute coronary syndromes, angina including stable angina and unstable angina, inflammation or inflammatory diseases including but not limited to vascular inflammation and dermal inflammation, congestive heart failure, coronary heart disease (CHD), hypertension, coronary ventricular arrythmias, surraventricular arrythmias, peripheral vascular disease, fatal myocardial infarction, non-fatal myocardial infarction, ischemia including cardiovascular ischemia, myocardium hybernation, transient ischemic attacks, ischemia unrelated to cardiovascular disease including ischemia-reperfusion injury such as injury due to hip surgery, knee surgery, organ transplant or percutaneous transluminal coronary angioplasty (PTCA), coronary reperfusion, restenosis, peri-operative (PCI) ischemic events, decreased need for revascularization, reduced infarct area, coagulation disorders, thrombocytopenia, deep vein thrombosis, pancreatitis, non-alcoholic steatohepatitis (NASH), diabetic neuropathy, retinopathy, painful diabetic neuropathy, psoriasis, critical limb ischemia, claudication, impotence, prostate cancer, hyperlipidemia, hyperlipoproteinemia, hypoalphalipoproteinemia, hypertriglyceridemia, any stenotic condition leading to ischemic pathology, diabetes including both Type I and Type II, ichtyosis, stroke, vulnerable plaques, vulnerable plaque rupture, Alzheimer's Disease, lower-limb ulceration, severe coronary ischemia, lymphomas, cataracts, endothelial dysfunction, xanthomas, end organ dysfunction, vascular disease, vascular disease that results from smoking and diabetes, carotid and coronary artery disease, regress and shrink established plaques, combinations of surgical procedures that result in endothelial injury, endothelial damage as a result of surgical procedures, morbidity associated with vascular disease, ulcerations in the arterial lumen, restenosis as a result of balloon angioplasty, and subindications of the foregoing.

The compositions and methods of the present invention may be used to increase HDL levels, increase low HDL levels, decrease LDL levels, decrease high LDL levels, temporarily increase LDL levels, decrease triglyceride levels, increase or decrease the level of other lipids, increase plaque stability or decrease the probability of plaque rupture, increase or decrease vasodilation, treat or prevent inflammation, treat or prevent inflammatory diseases or an inflammatory response, strengthen or stabilize smooth muscle and vessel intima, stimulate efflux of extracellular cholesterol for transport to the liver, modulate immune responses, mobilize cholesterol from atherosclerotic plaques, and modify any membrane, cell, tissue, organ, and extracellular region and/or structure in which compositional and/or functional modifications would be advantageous. The compositions and methods of the present invention also encompass topical applications and wound healing.

The methods generally comprise administering a liposome composition to a mammal, preferably a human having a disease or condition, which liposome composition comprises liposomes having an average diameter greater than 100 nm, preferably between 100 nm and 250 nm, preferably between 100 nm and 140nm, and more preferably between 110 nm and 120 nm. In particular embodiments, the liposomes administered to subjects have an average diameter size of between 100 and 110 nm, 10 and 120 nm, 120 and 130 nm, 130 and 140 nm, 140 and 150 nm, 150 and 160 nm, 160 and 170 nm, 170 and 180 nm, 180 and 190 nm, and between 190 and 200 nm. In a more preferred embodiment, the liposomes utilized are ETC-588 (a proprietary product of Esperion Therapeutics, Inc.) having an average diameter of between 100 nm and 140 nm, preferably between 110 nm and 120 nm after manufacture.

The present methods are particularly useful for treating atherosclerotic lesions as well as the other above-identified diseases and bodily conditions associated with lipid disorders. In one particular embodiment, the methods of the present invention may prophylactically inhibit or prevent the formation or expansion of atherosclerotic plaques, reduce the cholesterol content of atherosclerotic plaques, and/or reduce the volume of atherosclerotic plaques and hence the degree of any obstruction of the vascular lumen. The reduction in plaque volume will generally be at least 5%-30%, often as much as 50%, and in some instances 75% or more. The cholesterol content will generally be reduced by at least 10%-30%, often by 30%-50%, and in some instances as much as 75%-85% or more. Cholesterol may be mobilized from the plaques by either direct efflux into the liposomes or into lipoproteins that subsequently transfer the cholesterol to the liposomes. As cholesterol is transferred to the liposomes from the lipoproteins, the lipoproteins may receive more cholesterol from plaques. Generally, when cholesterol is received from lipoproteins, the cholesterol is transferred from HDL.

The methods may be useful to treat atherosclerosis as well as other disease and bodily conditions in a variety of animals and in a variety of blood vessels. Typically, the animal will be human, although non-human primates, dogs, cats, rodents, horses, cows, and the like may be treated by the methods of the present invention. Atherosclerosis of any blood vessel, such as the aorta, carotid arteries (common, internal, and external), coronary arteries, mesenteric arteries, renal arteries, iliac arteries, popliteal arteries, and the like, may be treated by the methods of the present invention. Likewise, phlebosclerosis or any venous condition in which deposits of plaques containing cholesterol or other material are formed within the intima or inner media of veins may be treated by the methods of the present invention. Human patients to be treated include infants, children, teenagers, adults, and the elderly who were not previously treated or those who were previously treated for cholesterol related disorders. The methods of the present invention also include treating patients prior to, during, or after surgery, and those with specific diseases or bodily conditions disclosed herein and/or other diseases and conditions not disclosed herein. In particular embodiments the methods include administering the liposome formulations described herein to patients suffering from arteriosclerosis including atherosclerosis, phlebosclerosis or any venous condition in which deposits of plaques containing cholesterol or other material are formed within the intima or inner media of veins, acute coronary syndromes, angina including stable angina and unstable angina, inflammation or inflammatory diseases including but not limited to vascular inflammation and dermal inflammation, congestive heart failure, coronary heart disease (CHD), hypertension, coronary ventricular arrythmias, surraventricular arrythmias, peripheral vascular disease, fatal myocardial infarction, non-fatal myocardial infarction, ischemia including cardiovascular ischemia, myocardium hybernation, transient ischemic attacks, ischemia unrelated to cardiovascular disease including ischemia-reperfusion injury such as injury due to hip surgery, knee surgery, organ transplant or PTCA, coronary reperfusion, restenosis, peri-operative (PCI) ischemic events, decreased need for revascularization, reduced infarct area, coagulation disorders, thrombocytopenia, deep vein thrombosis, pancreatitis, non-alcoholic steatohepatitis (NASH), diabetic neuropathy, retinopathy, painful diabetic neuropathy, psoriasis, critical limb ischemia, claudication, impotence, prostate cancer, hyperlipidemia, hyperlipoproteinemia, hypoalphalipoproteinemia, hypertriglyceridemia, any stenotic condition leading to ischemic pathology, diabetes including both Type I and Type II, ichtyosis, stroke, vulnerable plaques, vulnerable plaque rupture, Alzheimer's disease, lower-limb ulceration, severe coronary ischemia, lymphomas, cataracts, endothelial dysfunction, xanthomas, end organ dysfunction, vascular disease, vascular disease that results from smoking and diabetes, carotid and coronary artery disease, regress and shrink established plaques, combinations of surgical procedures that result in endothelial injury, endothelial damage as a result of surgical procedures, morbidity associated with vascular disease, ulcerations in the arterial lumen, restenosis as a result of balloon angioplasty, and subindications of the foregoing.

The liposome formulations of the present invention may also be administered to patients to increase HDL levels, increase low HDL levels, decrease LDL levels, decrease high LDL levels, temporarily increase LDL levels, decrease triglyceride levels, increase or decrease the level of other lipids, increase plaque stability or decrease the probability of plaque rupture, increase or decrease vasodilation, treat or prevent inflammation, treat or prevent inflammatory diseases or an inflammatory response, strengthen or stabilize smooth muscle and vessel intima, stimulate efflux of extracellular cholesterol for transport to the liver, modulate immune responses, mobilize cholesterol from atherosclerotic plaques, and modify any membrane, cell, tissue, organ, and extracellular region and/or structure in which compositional and/or functional modifications would be advantageous. The compositions and methods of the present invention also encompass topical applications and wound healing.

The methods of the present invention are also useful for prophylactic treatments, particularly to prevent relapse or complications in patients recovering from invasive vascular procedures. Vascular regions having injured endothelium are at increased risk for developing atherosclerotic plaques. Therefore, invasive vascular procedures, such as coronary angioplasty, vascular bypass grafting, and other procedures that injure the vascular endothelial layer, may be practiced in conjunction with the methods of the present invention. As the invasive procedure injures the endothelium, the liposomes act to remove cholesterol from the injured region and inhibit or prevent plaque formation of expansion during endothelial healing.

Hyperlipidemias may also be treated by the methods of the present invention. Administration of liposomes, alone or bound to apolipoprotein AI and apolipoprotein AII, apolipoprotein E, to individuals having hypoalphalipoproteinemia from genetic or secondary causes, familial combined hyperlipidemia, and familial hypercholesterolemia is a useful treatment.

The liposomes administered in the methods of the present invention will be composed of lipids as described above. The lipids will generally be in the liquid-crystalline state at 37° C. The lipids will also generally include one or more phospholipids, in some cases phosphatidylcholine or phosphatidylglycerol, although liposomes may be composed of many other lipids, examples of which are described above.

The liposomes may be administered in many ways. For example, the compositions may be administered orally or parenterally. Generally, the compositions will be administered parenterally, preferably intravenously or the administration may be intramuscular, subcutaneous, intraperitoneal, intra-arterial, intrathecal, via lymphatics, intravascular. Administration may be achieved via a chronically indwelling catheter or an acutely placed catheter via venous-infusion with a pump, via venous-infusion with a syringe, or via venous-infusion with a syringe-push administration. In other embodiments, the administration may be sublingual, buccal, mucosal, topical, rectal, vaginal, or transdermal. In a particular embodiment, the compositions of the present invention are administered topically to prevent or treat inflammation or to aid in the healing of wounds. In preferred embodiments, the liposomes will be administered intravenously. Often, the liposomes will be administered into a large central vein, such as the superior vena cava or inferior vena cava, to allow highly concentrated solutions to be administered into large volume and flow vessels. The liposomes may be administered intraarterially prior to, during, or following vascular procedures to deliver a high concentration directly to an affected vessel. The liposomes may also be administered directly to vessels in a topical manner by surgeons during open procedures. In some instances, the liposomes may be administered orally or transdermally. The liposomes may also be incorporated in vascular stents for long duration release following placement. This is particularly effective for angioplasty treatment of restenosis of lesions in the coronary arteries.

In particular embodiments, the liposomes formulations of the present invention may be administered intravenously, preferably via an infusion pump, at a rate of about: 1-2 ml/min, 2-3 ml/min, 3-4 ml/min, 4-5 ml/min, 5-6 ml/min, 6-7 ml/min, 7-8 ml/min, 8-9 ml/min, 9-10 ml/min, 10-11 ml/min, 11-12 ml/min, 12-13 ml/min, 13-14 ml/min, 14-15 ml/min, 15-16 ml/min, 16-17 ml/min, 17-18 ml/min, 18-19 ml/min, 19-20 ml/min, 20-30 ml/min, 30-40 ml/min, 40-50 ml/min, 50-60 ml/min, 60-70 ml/min, 70-80 ml/min, 80-90 ml/min, or 90-100 ml/min; or other predetermined rate of administration. In a particularly preferred embodiment, the liposome formulations of the present invention are administered intravenously via an infusion pump, by syringe pump, IV drip, and/or fast drip at a rate of about 10 ml/min. In another embodiment, the liposome formulations of the present invention may be administered via a dialysis or apheresis machine.

In other specific embodiments, the concentration of liposomes for intravenous infusion or other form of administration may be about: 1-10 mg/ml, 10-20 mg/ml, 20-30 mg/ml, 30-40 mg/ml, 40-50 mg/ml, 50-60 mg/ml, 60-70 mg/ml, 70-80 mg/ml, 80-90 mg/ml, 90-100 mg/ml, 100-110 mg/ml, 110-120 mg/ml, 120-130 mg/ml, 130-140 mg/ml, 140-150 mg/ml, 150-160 mg/ml, 160-170 mg/ml, 170-180 mg/ml, 180-190 mg/ml, 190-200 mg/ml, 200-210 mg/ml, 210-220 mg/ml, 220-230 mg/ml, 230-240 mg/ml, 240-250 mg/ml, 250-260 mg/ml, 260-270 mg/ml, 270-280 mg/ml, 280-290 mg/ml, 290-300 mg/ml, 300-310 mg/ml, 310-320 mg/ml, 320-330 mg/ml, 330-340 mg/ml, 340-350 mg/ml, 350-360 mg/ml, 360-370 mg/ml, 370-380 mg/ml, 380-390 mg/ml, or 390-400 mg/ml; or other predetermined concentration. In a preferred embodiment, the concentration of liposomes for intravenous infusion is about 200 mg/ml.

The methods of the present invention include administering the liposome formulations of the present invention therapeutically or prophylactically to animals having any of the above-mentioned diseases or bodily conditions or any other disease or bodily condition. The dose of liposomes may vary depending on the clinical condition and size of the subject or patient receiving treatment. In specific embodiments, the invention encompasses doses of about 20 mg/kg to about 300 mg/kg, 50 mg/kg to about 200 mg/kg, and/or doses that are tolerated by diseased patients effective for treatment while avoiding or reducing adverse effects.

In particular embodiments the liposomes of the present invention are administered to patients in single or divided doses between 50 mg/kg and 300 mg/kg. In separate preferred embodiments the liposomes of the present invention are administered to patients in single or divided doses between 100 mg/kg and 200 mg/kg, and more preferably between 150 mg/kg and 200 mg/kg. In other more particular embodiments, the vesicle particles are administered to patients in a single or divided doses of or between 110 mg/kg and 120 mg/kg, 120 mg/kg and 130 mg/kg, 130 mg/kg and 140 mg/kg, 140 mg/kg and 150 mg/kg, 150 mg/kg and 160 mg/kg, 160 mg/kg and 170 mg/kg, 170 mg/kg and 180 mg/kg, 180 mg/kg and 190 mg/kg, or between 190 mg/kg and 200 mg/kg. In other embodiments, the liposomes are administered to patients in fixed dose amounts of or between 0-1 g, 1-2 g, 2-3 g, 3-4 g, 4-5 g, 5-6 g, 6-7 g, 7-8 g, 8-9 g, 9-10 g, 10-11 g, 11-12 g, 12-13 g, 13-14 g, 14-15 g, 15-16 g, 16-17 g, 17-18 g, 18-19 g, or 19-20 g.

In specific embodiments, these dose amounts (or other dose amounts of liposomes) are administered in a single or divided dose in one or more intervals ranging from about: once a day, once every 2 days, once every 3 days, once every 4 days, once every 5 days, once every 6 days, once every 7 days, once 8 eight days, once every 9 days, once every 10 days, once every 11 days, once every 12 days, once every 13 days, once every 14 days, once every 2-3 weeks, once every 3-4 weeks, once every 4-5 weeks, once every 5-6 weeks, once every 6-7 weeks, once every 7-8 weeks, once every 2-3 months, once 3-4 months, once every 4-5 months, once every 5-6 months, once every 6-7 months, once every 7-8 months, once every 8-9 months, once every 9-10 months, once every 10-11 months, once every 11-12 months, once every 1-2 years, once every 2-3 years, once every 3-4 years, once every 4-5 years; or otherwise administered at predetermined time intervals for a predetermined treatment period. In a particularly preferred embodiment the time interval between dose amounts during a course of therapy is about once a week.

Preferred treatment periods for a course of therapy may span from the time when the first dose is administered to about: 1 day, 2 days, 3 days, 4 days, 5 days, 6 days, 7 days, 1-2 weeks, 2-3 weeks, 3-4 weeks, 4-5 weeks, 5-6 weeks, 6-7 weeks, 7-8 weeks, 2-3 months, 3-4 months, 4-5 months, 5-6 months, 6-7 months, 7-8 months, 8-9 months, 9-10 months, 10-11 months, 11-12 months, 1-2 years, 2-3 years, 3-4 years, and 4-5 years.

In certain embodiments, a course of therapy may be cycled or intermittent where the compounds and formulations of the present invention are administered to subjects for a finite period of time or until adverse events or side effects warrant reduction in dose or cessation of treatment for a particular period of time (rest period). Reduction in dose, cessation in treatment, or length of rest period may be determined by the discretion of a physician. After such a rest period, or after such adverse events or other side effects have subsided, or at the discretion of the physician, treatment may be resumed if needed.

Tolerability, safety, and effectiveness (including but not limited to cholesterol levels, liver enzymes, blood levels of specific determinants or markers, and physiological factors) may be monitored prior to, during, or following a course of therapy and/or during the various periods during an intermittent or cyclic course of therapy. In preferable embodiments, physiological factors, vascular changes, stability of plaques, vessel walls, and other markers are monitored by imaging techniques including but not limited to (e.g. magnetic resonance imaging (MRI), intravascular ultrasound, and blood flow techniques). Blood markers may be monitored by blood tests known in the art. During or after completion of such monitoring, the treatment regimen may be modified accordingly [including altering the dose amounts, size or distribution of liposomes, rates of administration, concentration of liposomes, number of doses, time between intervals, or length of treatment period(s)].

In addition, the liposomes of the present invention are administered to patients in combination with other drugs, therapeutics, or lipid regulating therapies. For example, the liposomes of the present invention may be administered concomitantly with HMG-CoA reductase inhibitors, fibrates, bile acid sequestrants, nicotinic acid and other anti-hyperlipidemic agents, antidiabetic agents and/or glycemic control agents, anti-inflammatory agents, antihypertensive agents, anti-coagulation agents, Apo-AI mimetics and HDL elevators of Esperion Therapeutics Inc., other cardiovascular agents known in the art, and combinations thereof.

In one particular embodiment, the liposomes of the present invention are administered to a patients suffering from diabetes concomitantly with other anti-diabetic and/or glycemic control agents. In other particular embodiments, the liposomes are administered to prevent, treat, or manage a patient suffering from inflammation, an inflammatory disease, or to prevent or reduce an inflammatory response. In other specific embodiments, the liposomes of the present invention are topically applied to the skin to treat cutaneous, subcutaneous, or localized inflammation, or to aid in the healing of a wound. In another specific embodiment, the liposomes of the present invention are administered to a patient to prevent, manage, or treat a patient afflicted by Alzheimer's disease. In another specific embodiment, the liposomes of the present invention are administered to a patient to prevent, manage, or treat a patient suffering from atherosclerosis, phlebosclerosis, or any condition in which deposits of plaques containing cholesterol or other material are formed within the intima or inner media of blood vessels. In another specific embodiment, the liposomes of the present invention are administered to a patient to prevent, manage, or treat a patient suffering from ischemia including non-cardiovascular ischemia. In another specific embodiment, the liposomes of the present invention are used to pretreat patients prior to elective surgery that may induce ischemia reperfusion injury, such as hip surgery, knee surgery, organ transplant, PTCA.

In a preferred embodiment, the treatment period for a course of therapy is no longer than 14 weeks. In other particularly preferred embodiments, liposomes are administered from 5-200 mg/kg at 4-7 day intervals with either 1-4, 1-8, or 1-14 total doses given during each course of therapy. In another particular embodiment, liposomes are administered 5-200 mg/kg once a week for about 4 to 16 weeks preferably about 10 weeks (for a total of 10 treatments).

In one specific embodiment liposomes in a concentration of 50-200 mg/ml having an average diameter between 50-250 nm±50%, preferably between 100-140 nm±50%, more preferably between 110-120 nm±50% are administered to a patient suffering from atherosclerosis alone or in combination with other anti-sclerotic agents, in a dose amount of 100 mg/kg, and at a rate of 10 ml/min or faster every 7 days for a total of 10 doses.

In another specific embodiment liposomes in a concentration of 50-200 mg/ml having an average diameter between 50-250 nm±50%, preferably between 100-140 nm±50%, more preferably between 110-120 nm±50% are administered to a patient suffering from atherosclerosis alone or in combination with other anti-sclerotic agents, in a dose amount of 150 mg/kg, and at a rate of 10 ml/min or faster every 7 days for a total of 12 doses.

In another specific embodiment liposomes in a concentration of 50-200 mg/ml having an average diameter between 50-250 nm±50%, preferably between 100-140 nm±50%, more preferably between 110- 120 nm±50% are administered to a patient suffering from atherosclerosis alone or in combination with other anti-sclerotic agents, in a dose amount of 200 mg/kg, and at a rate of 10 ml/min or faster every 7 days for a total of 14 doses.

In another embodiment liposomes in a concentration of 50-200 mg/ml having an average diameter between 50-250 nm±50%, preferably between 100-140 nm±50%, more preferably between 110-120 nm±50% are administered to a patient suffering from angina congestive heart failure, coronary heart disease, hypertension, or arrythmias alone or in combination with other cardiovascular agents, in a dose amount of 100 mg/kg, and at a rate of 10 ml/min or faster every 7 days for a total of 10 doses.

In another embodiment liposomes in a concentration of 50-200 mg/ml having an average diameter between 50-250 nm±50%, preferably between 100-140 nm±50%, more preferably between 110-120 nm±50% are administered to a patient suffering from angina congestive heart failure, coronary heart disease, hypertension, or arrythmias alone or in combination with other cardiovascular agents, in a dose amount of 150 mg/kg, and at a rate of 10 ml/min or faster every 7 days for a total of 12 doses.

In another embodiment liposomes in a concentration of 50-200 mg/ml having an average diameter between 50-250 nm±50%, preferably between 100-140 nm±50%, more preferably between 110-120 nm±50% are administered to a patient suffering from angina congestive heart failure, coronary heart disease, hypertension, or arrythmias alone or in combination with other cardiovascular agents, in a dose amount of 200 mg/kg, and at a rate of 10 ml/min or faster every 7 days for a total of 14 doses.

In another embodiment liposomes in a concentration of 50-200 mg/ml having an average diameter between 50-250 nm±50%, preferably between 100-140 nm±50%, more preferably between 110-120 nm±50% are administered to a patient suffering from inflammation alone or in combination with other anti-inflammatory agents, in a dose amount of 100 mg/kg, and at a rate of 10 ml/min or faster every 7 days for a total of 10 doses.

In another embodiment liposomes in a concentration of 50-200 mg/ml having an average diameter between 50-250 nm±50%, preferably between 100-140 nm±50%, more preferably between 1 10-120 nm±50% are administered to a patient suffering from inflammation alone or in combination with other anti-inflammatory agents, in a dose amount of 150 mg/kg, and at a rate of 10 ml/min or faster every 7 days for a total of 12 doses.

In another embodiment liposomes in a concentration of 50-200 mg/ml having an average diameter between 50-250 nm±50%, preferably between 100-140 nm±50%, more preferably between 110-120 nm±50% are administered to a patient suffering from inflammation alone or in combination with other anti-inflammatory agents, in a dose amount of 200 mg/kg, and at a rate of 10 ml/min or faster every 7 days for a total of 14 doses.

In another embodiment liposomes in a concentration of 50-200 mg/ml having an average diameter between 50-250 nm±50%, preferably between 100-140 nm±50%, more preferably between 110-120 nm±50% are administered to a patient suffering from ischemia alone or in combination with anti-ischemic agents, in a dose amount of 100 mg/kg, and at a rate of 10 ml/min or faster every 7 days for a total of 10 doses.

In another embodiment liposomes in a concentration of 50-200 mg/ml having an average diameter between 50-250 nm±50%, preferably between 100-140 nm±50%, more preferably between 110-120 nm±50% are administered to a patient suffering from ischemia alone or in combination with anti-ischemic agents, in a dose amount of 150 mg/kg, and at a rate of 10 ml/min or faster every 7 days for a total of 12 doses.

In another embodiment liposomes in a concentration of 50-200 mg/ml having an average diameter between 50-250 nm±50%, preferably between 100-140 nm±50%, more preferably between 110-120 nm±50% are administered to a patient suffering from ischemia alone or in combination with anti-ischemic agents, in a dose amount of 200 mg/kg, and at a rate of 10 ml/min or faster every 7 days for a total of 14 doses.

In another embodiment liposomes in a concentration of 50-200 mg/ml having an average diameter between 50-250 nm±50%, preferably between 100-140 nm±50%, more preferably between 110-120 nm±50% are administered to a patient suffering from hyperlipidemia, hyperlipoproteinemia, hypoalphalipoproteinemia, hypertriglyceridemia alone or in combination with antihyperlipidemic, antihyperlipoproteinemic, antihypoalphalipoproteinemic, or antihypertriglyceridemic agents, in a dose amount of 100 mg/kg, and at a rate of 10 ml/min or faster every 7 days for a total of 10 doses.

In another embodiment liposomes in a concentration of 50-200 mg/ml having an average diameter between 50-250 nm±50%, preferably between 100-140 nm±50%, more preferably between 110-120 nm±50% are administered to a patient suffering from hyperlipidemia, hyperlipoproteinemia, hypoalphalipoproteinemia, hypertriglyceridemia alone or in combination with antihyperlipidemic, antihyperlipoproteinemic, antihypoalphalipoproteinemic, or antihypertriglyceridemic agents, in a dose amount of 150 mg/kg, and at a rate of 10 ml/min or faster every 7 days for a total of 12 doses.

In another embodiment liposomes in a concentration of 50-200 mg/ml having an average diameter between 50-250 nm±50%, preferably between 100-140 nm±50%, more preferably between 110-120 nm±50% are administered to a patient suffering from hyperlipidemia, hyperlipoproteinemia, hypoalphalipoproteinemia, hypertriglyceridemia alone or in combination with antihyperlipidemic, antihyperlipoproteinemic, antihypoalphalipoproteinemic, or antihypertriglyceridemic agents, in a dose amount of 200 mg/kg, and at a rate of 10 ml/min or faster every 7 days for a total of 14 doses.

In another embodiment liposomes in a concentration of 50-200 mg/ml having an average diameter between 50-250 nm±50%, preferably between 100-140 nm±50%, more preferably between 110-120 nm±50% are administered to a patient suffering from cardiovascular disease and diabetes alone or in combination with other antidiabetic and/or glycemic control agents and/or cardiovascular agents, in a dose amount of 100 mg/kg, and at a rate of 10 ml/min or faster every 7 days for a total of 10 doses.

In another embodiment liposomes in a concentration of 50-200 mg/ml having an average diameter between 50-250 nm±50%, preferably between 100-140 nm±50%, more preferably between 110-120 nm±50% are administered to a patient suffering from cardiovascular disease and diabetes alone or in combination with other antidiabetic and/or glycemic control agents and/or cardiovascular agents, in a dose amount of 150 mg/kg, and at a rate of 10 ml/min or faster every 7 days for a total of 12 doses.

In another embodiment liposomes in a concentration of 50-200 mg/ml having an average diameter between 50-250 nm±50%, preferably between 100-140 nm±50%, more preferably between 110-120 nm±50% are administered to a patient suffering from cardiovascular disease and diabetes alone or in combination with other antidiabetic and/or glycemic control agents and/or cardiovascular agents, in a dose amount of 200 mg/kg, and at a rate of 10 ml/min or faster every 7 days for a total of 14 doses.

Dose regimes also include bolus administrations or continuous infusion treatment that may include the use of a primer dose followed by a maintenance dose. In preferred embodiments, the dose will be constant over the course of treatment. In other preferred embodiments, the dose will vary. In particular embodiments, the liposome formulations will be administered only once, and in other embodiment serially administered in multiple doses and in other embodiments administered in non-consecutive multiple doses. The duration, schedule of treatments, and dosing regimens may be varied by methods well known to those skilled in the art. Serum measurements of total free cholesterol, total esterified cholesterol, HDL cholesterol, LDL cholesterol, and VLDL cholesterol may be used to assess and modify dosage amounts and schedules during the treatment regimen. As cholesterol is mobilized from plaques, total serum cholesterol rises. It is desirable that total serum cholesterol and HDL cholesterol rise during therapy and esterified cholesterol drop (or be minimally affected) during therapy. The liposome dose for different animals will generally approximate the human weight-determined dosage. Patients treated with such compositions and according to such methods may be of any age and may be afflicted with one or more of the diseases or bodily conditions enumerated above and/or other diseases and conditions.

### EXAMPLES

The following examples are offered by way of illustration and not limitation.

### Example 1

### Multiple Doses of ETC-588 Liposomes in Patients With Atherosclerosis

A multiple-dose study was conducted which evaluated the effects of multiple-doses of ETC-588 liposomes (the ETC-588-003 study) in human patients with atherosclerosis. ETC-588 liposomes (200 mg/ml) were infused intravenously using an infusion pump. ETC-588 in plasma was assayed as phospholipid (PL). The dose groups were as follows: placebo (7 or 14 doses), 50 mg/kg (14 doses), 100 mg/kg (7 doses) or 200 mg/kg (7 doses) were studied. Doses were given at 4 day intervals (q4d) or 7 day intervals (q7d). A total of 42 patients participated in the study (male: 36, female: 6) between 44 and 76 years of age (the mean age being 63±7 years). The mean weight of the patients was 87.7 kg±17.0 kg with a range of 52.4 kg to 147.7 kg. The mean baseline HDL-cholesterol levels of the patients were 36±4 mg/dL and the mean baseline total cholesterol levels of the patients were 182±36 mg/dL. In addition the following percentage of patients had a cardiovascular history of: coronary artery disease: 98%, peripheral artery disease: 2%; coronary artery bypass graft: 57%; Stable Angina: 29%; and Unstable Angina: 19%.

Total and unesterified cholesterol (UC) and PL were assayed by standard, automated methods. Subjects were allocated to treatment groups according to a randomization schedule. Safety and tolerability, laboratory data, vital signs and adverse events were summarized at each time point and for pre-dose to post-dose change using descriptive statistics. Pharmacokinetic and pharmacodynamic measures were summarized using descriptive statistics. Of the 42 patients who participated in the study 36 actually received the drug although 2 patients withdrew before completing all doses for reasons other than adverse events or side effects. The number of patients who reported any adverse event (AE) was 26 total (72%) and the number of patients who reported a serious adverse event (SAE) was 5 (14%) in total. The number of patients included in the safety analysis was 36 while 33 patients were included in the pharmacokinetic and pharmacodynamic analyses (with incomplete pharmacokinetic data for 3 patients). The effects of ETC-588 on endothelial function (vascular structure), inflammatory markers and magnetic resonance imaging (MRI) was also examined.

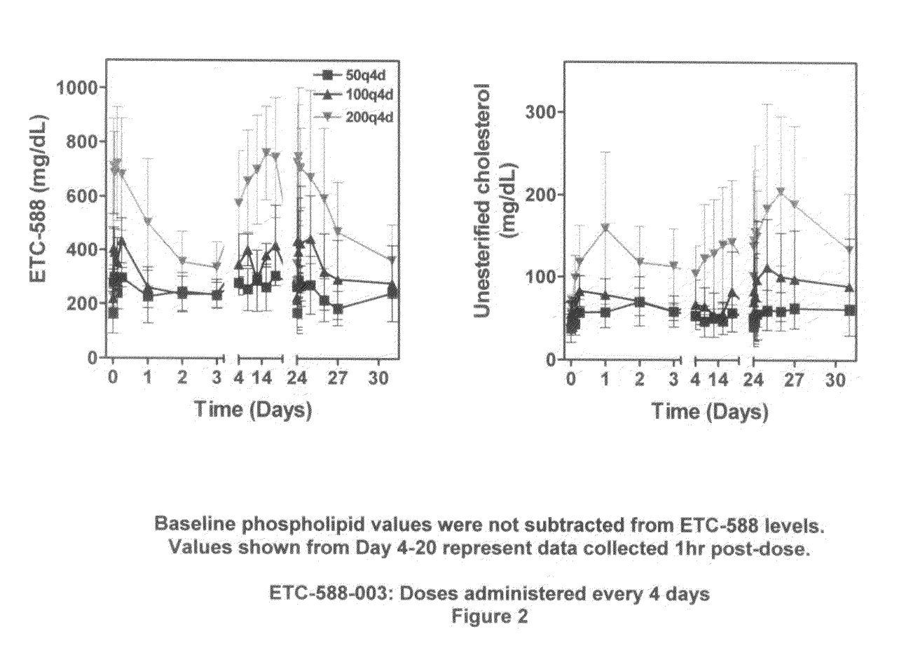

The results are presented in FIGS. 2 & 3. The results demonstrate that cholesterol mobilization occurred across all doses of ETC-588 with varying efficiencies. The following adverse events and serious adverse events were reported for each group:

Dose Group

| Adverse | | 50 | 50 | 100 | 100 | 200 | 200 |
|---|---|---|---|---|---|---|---|
| Event | | mg/kg | mg/kg | mg/kg | mg/kg | mg/kg | mg/kg |
| Category | Placebo | q7d | q4d | q7d | q4d | q7d | q4d |
| (AE) | (n = 6) | (n = 4) | (n = 4) | (n = 5) | (n = 5) | (n = 5) | (n = 7) |
| Any AE | 4 | 4 | 2 | 5 | 3 | 2 | 6 |
| | (66.7%) | (100%) | (50.0%) | (100%) | (60.0%) | (40.0%) | (85.7%) |
| Any SAE | 0 | 0 | 1 | 0 | 1 | 1 | 2 |
| | | | (25.0%) | | (25.0%) | (20.0%) | (28.6%) |
| Any | 0 | 0 | 0 | 0 | 0 | 0 | 0 |
| Treatment | | | | | | | |
| Ceased | | | | | | | |

The most common adverse events in patients with at least once occurrence of an adverse event are summarized below. Serious adverse events (bladder neck contracture, chest pain, hyperglycemia or exacerbation of diabetes) was considered by the treating physician to be unrelated to the administration of ETC-588:

| Adverse Event | | | | |
|---|---|---|---|---|
| (Total number of | Placebo | 50 mg/kg | 100 mg/kg | 200 mg/kg |
| incidences) | N = 6 | N = 8 | N = 10 | N = 12 |
| Headache | 2 | 1 | 5 | 4 |
| (23) | | | | |
| Dizziness | 1 | 3 | 0 | 0 |
| (6) | | | | |
| Fatigue | 0 | 0 | 2 | 3 |
| (6) | | | | |
| Nausea | 0 | 2 | 0 | 1 |
| (5) | | | | |
| Blood | 0 | 2 | 1 | 0 |
| Pressure | | | | |
| Increase | | | | |
| (4) | | | | |
| Chest Pain | 0 | 1 | 1 | 0 |
| (3) | | | | |

Overall, the results from the ETC-588-003 study suggest that ETC-588 liposomes mobilize cholesterol in a dose-dependent manner and that doses of 200 mg/kg or less are generally safe and well-tolerated. Further, as demonstrated above, seven day dosing intervals appear to be optimal over 4 day dosing intervals for ETC-588 liposomes because 7 day dosing enables optimal clearance of phospholipid and unesterified cholesterol in serum. In addition, 7 day dosing intervals may be optimal over 4 day dosing intervals because adverse events and serious adverse events may be reduced or prevented.

For example, as demonstrated in the tables above, when 200 mg/kg was administered in 7 day intervals the percentage of any adverse event was 40% and any serious adverse event was 20%. In contrast when 200 mg/kg was administered in 4 day intervals the percentage of any adverse event was 85.7% and any serious adverse event was 28.6%. Similarly, when 100 mg/kg was administered in 7 day intervals the percentage of “any serious adverse event” was 0%. In contrast, when 100 mg/kg was administered in 4 day intervals the percentage of “any serious adverse event” was 25%. Likewise, when 50 mg/kg was administered in 7 day intervals the percentage of “any serious adverse event” was 0%. However, when 50 mg/kg was administered in 4 day intervals the percentage of “any serious adverse event” was 25%.

What is claimed is:

1. A method for treating or managing a disease or bodily condition associated with a lipid disorder, comprising administering liposomes to a subject in need thereof in a single or divided dose of between about 50 mg/kg and 300 mg/kg, said liposomes comprising phospholipids and an aqueous layer, having an average diameter between about 50 nm and 250 nm, and are administered according to a dosing schedule.

2. The method of claim 1 wherein the dosing schedule having an interval of 4 to 10 days.

3. The method of claim 1 wherein the liposomes have an average diameter of between about 50 and 150 nm.

4. The method of claim 1 wherein the liposomes have an average diameter of between about 50 and 200 nm.

5. The method of claim 1 wherein the liposomes have an average diameter of between about 100 and 200 nm.

6. The method of claim 1 wherein the liposomes have an average diameter of between about 100 and 250 nm.

7. The method of claim 1 wherein the liposomes have an average diameter of between about 150 and 200 nm.

8. The method of claim 1 wherein the liposomes have an average diameter of between about 150 and 250 nm.

9. The method of claim 1, wherein the liposomes are administered in combination with other phospholipid vesicles selected from the group consisting of multilamellar vesicles, large unilamellar vesicles, small unilamellar vesicles, and mixtures thereof.

10. The method of claim 1 which does not cause an increase in the LDL levels of the subject.

11. The method of claim 1 which does cause a decrease in the LDL levels of the subject.

12. The method of claim 1 in which the phospholipid is selected from the group consisting of egg phosphatidylcholine, egg phosphatidylglycerol, distearoylphosphatidylcholine, distearoylphosphatidylglycerol, phosphatidylcholine, phosphatidylglycerol, lecithin, beta, gamma-dipalmitoyl-alpha-lecithin, sphingomyelin, phosphatidylserine, phosphatidic acid, N-(2,3-di(9-(Z)-octadecenyloxy))-prop-1-yl-N,N,N-trimethylammonium chloride-phosphatidylethanolamine, lysolecithin, lysophosphatidylethanolamine, phosphatidylinositol, cephalin, cardiolipin, cerebroside, diacetylphosphate, dioleoylphosphatidylcholine, dipalmitoylphosphatidylcholine, dipalmitoylphosphatidylglycerol, dioleoylphosphatidylglycerol, palmitoyl-oleoyl-phosphatidylcholine, di-stearoyl-phosphatidylcholine, stearoyl-palmitoyl-phosphatidylcholine, di-palmitoyl-phosphatidylethanolamine, di-stearoyl-phosphatidylethanolamine, di-myrstoyl-phosphatidylserine, di-oleoyl-phosphatidylcholine, oleoyl-palmitoyl phosphatidylcholine, and mixtures thereof.

13. The method of claim 1 wherein the phospholipid is palmitoyl-oleoyl phosphatidylcholine.

14. The method of claim 1 wherein the disease or bodily condition is selected from the group consisting of atherosclerosis, phlebosclerosis or any venous condition in which deposits of plaques containing cholesterol or other material are formed within the intima or inner media of veins, acute coronary syndromes, angina including, stable angina, unstable angina, inflammation, vascular inflammation, dermal inflammation, congestive heart failure, coronary heart disease (CHD), ventricular arrythmias, peripheral vascular disease, myocardial infarction, onset of fatal myocardial infarction, non-fatal myocardial infarction, ischemia, cardiovascular ischemia, transient ischemic attacks, ischemia unrelated to cardiovascular disease, ischemia-reperfusion injury, decreased need for revascularization, coagulation disorders, thrombocytopenia, deep vein thrombosis, pancreatitis, non-alcoholic steatohepatitis, diabetic neuropathy, retinopathy, painful diabetic neuropathy, claudication, psoriasis, critical limb ischemia, impotence, hyperlipidemia, hyperlipoproteinemia, hypoalphalipoproteinemia, hypertriglyceridemia, any stenotic condition leading to ischemic pathology, diabetes including both Type I and Type II, ichtyosis, stroke, vulnerable plaques, Alzheimer's disease, lower-limb ulceration, severe coronary ischemia, lymphomas, cataracts, endothelial dysfunction, xanthomas, end organ dysfunction, vascular disease, vascular disease that results from smoking and diabetes, carotid and coronary artery disease, regress and shrink established plaques, unstable plaques, vessel intima that is weak, unstable vessel intima, endothelial injury, endothelial damage as a result of surgical procedures, morbidity associated with vascular disease, ulcerations in the arterial lumen, restenosis as a result of balloon angioplasty, and subindications of the foregoing.

15. The method of claim 1 wherein the treatment strengthens the vessel wall intima, stimulates efflux of extracellular cholesterol for transport to the liver, modulates immune responses, mobilizes cholesterol from atherosclerotic plaques, aids in wound healing, modifies any bodily membrane, cell, tissue, organ, extracellular region, or structure.

16. The method of claim 1 wherein the liposomes are administered in combination with or are adjunctively bound to compounds selected from the group consisting of peptides, paraoxonase, lipoprotein lipase, Apo A-I, Apo A-I mimetics, variants of A-I, and combinations thereof.

17. The method of claim 1 wherein the liposomes are administered in combination with small molecules or drugs that affect cholesterol levels.

18. The method of claim 17 wherein the small molecule is a statin, reconstituted HDL, small HDL, or synthetic mimetic HDL lipoprotein particle.

19. The method of claim 1 wherein the liposomes are administered in combination with one or more cardiovascular agents, anti-diabetic agents, or other therapeutic substances.

20. The method of claim 19 wherein the cardiovascular agent is selected from the group consisting of small molecules, statins, aspirin, beta-blockers including clopidodrel, calcium blockers, heparin, including low molecular weight heparin, glucose lowering agents, nitrates, IIb/IIIa inhibitors, ACE inhibitors, fibrates, and bile acid sequestrants.

21. The method of claim 1 wherein the liposomes are administered in a dose of about 50 mg/kg to about 300 mg/kg every 7 days.

22. The method of claim 1 wherein the liposomes are administered once.

23. The method of claim 1 wherein the liposomes are administered 2 to 14 times at 4-7 day intervals.

24. The method of claim 1 wherein the liposomes are administered 2-14 times at weekly or biweekly intervals.

25. The method of claim 1 wherein the liposomes are administered 2-14 times at 1 month to 11 month intervals.

26. The method of claim 1 wherein the liposomes are administered 2-14 times at 3 or 4 year intervals.

27. A method of reducing cholesterol in peripheral tissue while reducing or avoiding adverse effects associated with liposome therapy which comprises administering to a patient in need thereof a dose of between 100 mg/kg to 200 mg/kg of liposomes having an average particle size between 50 nm and 250 nm.

28. A method of preventing or treating a disease or disorder associated with abnormal cholesterol levels which comprises administering to a patient in need thereof doses of liposomes from 100 mg/kg to 200 mg/kg, said administration being made in a single or multiple dose every 7 or more days.

29. The method of claim 28 wherein the liposomes are administered 6 to 14 times.

30. A method of reducing cholesterol in a diseased human which comprises administering to said human liposomes in a dose of about 100 mg/kg to about 200 mg/kg every 4 or more days, said liposomes having an average diameter of between 50 nm and 250 nm.

31. The method of claim 30 further comprising administering said liposomes in combination with one or more cardiovascular agents, antidiabetic and/or glycemic control agents, and/or other therapeutic agents.

32. The method of claim 1, wherein the disease is atherosclerosis.
